(19) United States
(12) Patent Application Publication
(10) Pub. No.: US 2007/0110802 A1
(43) Pub. Date: May 17, 2007

(54) Wet granulation process

(76) Inventors: Janan Jona, Simi Valley, CA (US); Sekhar Kanapuram, Thousand Oaks, CA (US); Dominick Daurio, Thousand Oaks, CA (US)

Correspondence Address: AMGEN INC., MAIL STOP 28-2-C, ONE AMGEN CENTER DRIVE, THOUSAND OAKS, CA, 91320-1799, US

(21) Appl. No.: 11/599,924

(22) Filed: Nov. 14, 2006

Related U.S. Application Data
(60) Provisional application No. 60/737,370, filed on Nov. 15, 2005.

(51) Int. Cl. A61K 9/20; B27N 3/00

(52) U.S. Cl. 424/464; 264/109

(57) ABSTRACT

The present invention relates to a process of wet granulation, and specifically wet granulation of material that is susceptible to convert from anhydrous form to hydrate form.

This application claims the benefit of U.S. Provisional Application No. 60/737,370, filed Nov. 15, 2005, which is hereby incorporated herein by reference in their entireties.

### FIELD OF THE INVENTION

The present invention relates to a process of wet granulation, and specifically wet granulation of material that can convert physical forms.

### BACKGROUND OF THE INVENTION

The pharmaceutical industry employs various methods for compounding pharmaceutical agents in tablet formulations. In particular, wet granulation is one of the most prevalent methods. Wet granulation methods can be used where the flow properties of a compound such as an active pharmaceutical ingredient (“API”) are poor which result in content uniformity issues when formulated as a dry blend. Wet granulation is commonly used to improve the processing characteristics of a powder blend, including improved flowability, content uniformity and more uniform particle size. The use of water in wet granulation may cause chemical degradation or physical form conversion. Herman et al., Int. J. of Pharm., 55:143-146 (1989) describe property changes associated with the conversion of anhydrous to the hydrate form of theophyllin. Morris et al. describe analytical methods available to monitor humidity related changes in an API (Int. J. of Pharm., 108:195-206 (1994)). WO 01/02365 describes API hydrate formation prior to the wet granulation of the API hydrate. Airaksinen et al., J. Pharm. Sci., 92:516-528 (2003) describe methods of addressing hydrate formation during wet granulation with theophyllin. Jurgensen et al., Pharm. Res., 19:1285-1291 (2002) describe analytical methods for monitoring hydrate formation during wet granulation of caffeine and theophyllin. WO 01/21211 describes a method for lessening polymorphic conversion of a drug. U.S. Pat. No. 6,224,907 describes including a solubility enhancing agent to maintain alkaline conditions in a dosage form.

Although it is known that controlling the pH may increase the solubility of an API, there has been no mention of controlling the pH during wet granulation to minimize the formation of hydrates.

### DESCRIPTION OF THE DRAWINGS

FIG. 1 shows the XRPD results from wet granulation material wetted with de-ionized water as described in Example 1. The anhydrous material standard and placebo materials are included. The predominant XRPD peaks for the anhydrous material occur at 2 theta: 8.924, 21.910, and 24.442. The predominant XRPD peaks for the granulated material with Compound A occurs at 2 theta: 8.424, 11.173, and 16.756, peaks not observed in the placebo material. These results indicate that the granulated material with API changes from the anhydrous form during the wet granulation step. Similar patterns were observed for Examples 2-5. XRPD analysis was performed on a Philips X'Pert instrument.

FIG. 2 shows the XRPD results from wet granulation material wetted with 1 M sodium acetate buffer (pH 4) as described in Example 6. The anhydrous material standard and placebo materials are included. The predominant XRPD peaks for the anhydrous material occur at 2 theta: 8.924, 21.910, and 24.442. The predominant XRPD peaks for the anhydrous peaks at 2 theta: 8.924, 21.910, and 24.442 were observed for the pH 4 material, peaks not observed in the placebo material. These results indicate that the granulated material with API does not change from the anhydrous form during the buffered wet granulation step.

FIG. 3 shows the XRPD results for the anhydrous form standard and the dihydrate form standard. The predominant XRPD peaks for the anhydrous material occur at 2 theta: 8.924, 21.910, and 24.442. The predominant XRPD peaks for the dihydrate material occur at 2 theta: 8.424, 11.173, and 16.756.

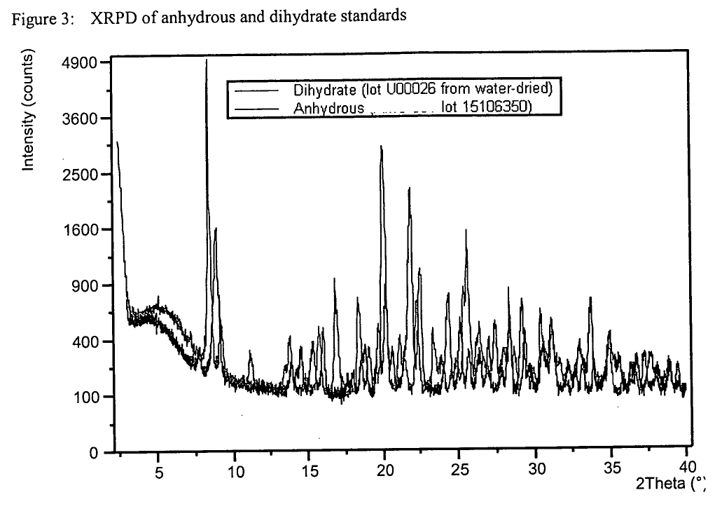

FIG. 4 shows the differential scanning calorimetry (DSC) results for the anhydrous form standard and the dihydrate form standard. The anhydrous material displays an endothermic event at about 275° C. The dihydrate form displays two endothermic events, the first at about 100° C. and the second at about 275° C.

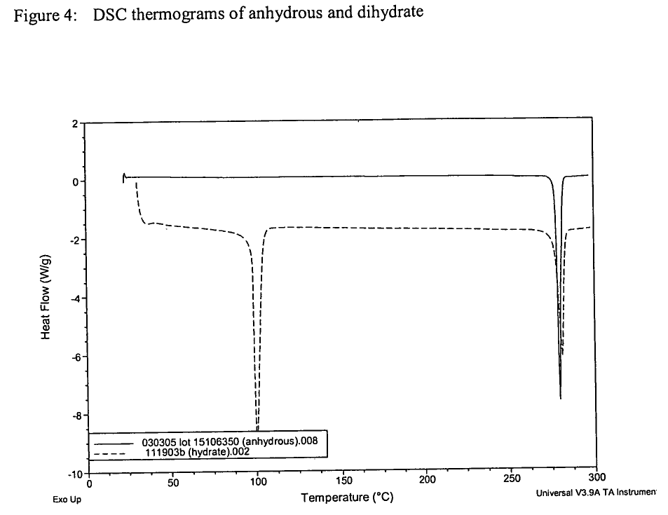

FIG. 5 shows the DSC results for wet granulation wetted with de-ionized water as described in Example 1. The anhydrous material is a standard and the placebo granulation mix does not have compound A. The anhydrous material displays an endothermic event at about 275° C. Both wet granulated materials displayed endothermic peaks at about 150° C.

FIG. 6 shows the DSC results from wet granulation material wetted with 1 M sodium acetate buffer (pH 4) as described in Example 6 and a placebo granulation mix without compound A. The anhydrous material is a standard. The anhydrous material displays an endothermic event at about 275° C. Both wet granulated materials displayed endothermic peaks at about 150° C.

FIG. 7 shows the DSC results from wet granulation material wetted with 1 M sodium acetate buffer (pH 4) as described in Example 6, results for wet granulation wetted with de-ionized water as described in Example 1 and standard dihydrate form. The dihydrate form displays two endothermic events, the first at about 100° C. and the second at about 275° C. Both wet granulated materials displayed endothermic peaks at about 150° C.

FIG. 8 shows the Raman spectroscopy results for the anhydrous form standard and the dihydrate form standard. The anhydrous material displays 2 peaks between about 1420-1440 cm⁻¹. The dihydrate material has a peak at about 1460 cm⁻¹.

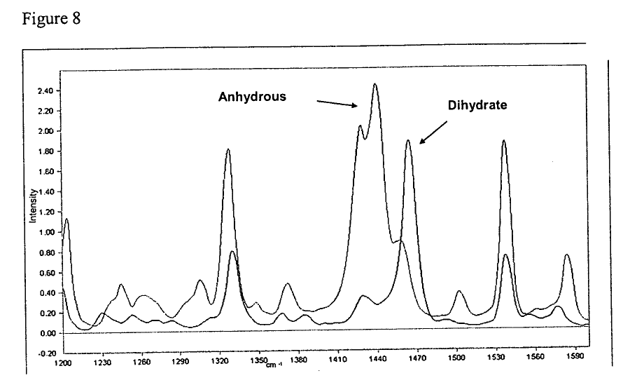

FIG. 9 shows the pH dependent solubility chart of Compound A. The solubility increases at a pH consistent with the approximate pKa value.

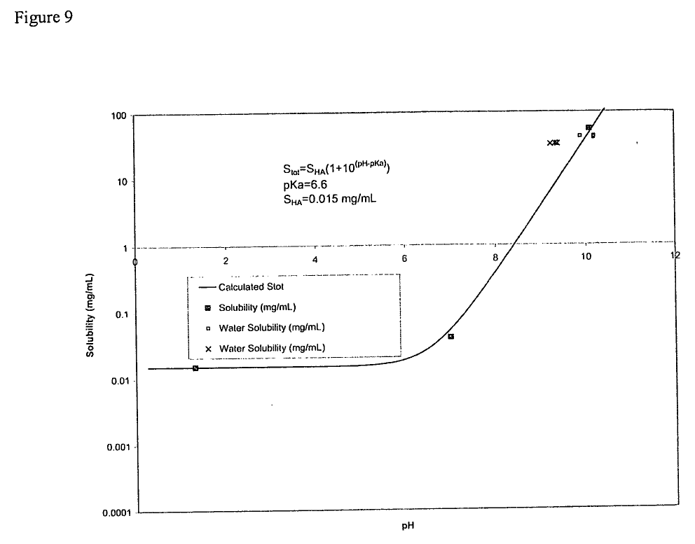

### DESCRIPTION OF THE INVENTION

The present invention is generally directed to a method of preventing physical form change of a compound during a wet granulation process. The results obtained by the inventors indicate a surprising adjustment of the pH dramatically changes the conversion of physical forms.

The invention also relates to a method of the present invention wherein the pH adjustment is made with an acidic or basic buffer. The invention also relates to a method of the present invention wherein the acidic buffer is selected from (where the range represents the effective range of each buffer):
Glutamate: as buffer between pH 2.0-4.0;
Citrate: as buffer between pH 2.0-6.0;
Acetate: as buffer between pH 3.8-5.8;
Carbonate: as buffer between pH 5.4-7.0; and
Phosphate: as buffer between pH 6.2-7.0.
   The invention also relates to a method of the present invention wherein the basic buffer is selected from (where the range represents the effective range of each buffer):
Glutamate: as buffer between pH 8.7-10.7;
Phosphate: as buffer between pH 7.0-8.2;
Carbonate: as buffer between pH 7.0-7.4; and
Triethanolamine: as buffer between pH 7.0-9.0.
   Buffers in general are known in the art (see for example, Pharmaceutical Dosage Forms: Parenteral Medications, 2^{nd} ed., K. Avis, H. Lieberman, and L. Lachman eds., 1:198 (1992); or the CRC Handbook of Chemistry and Physics, 83rd ed., David Lide ed., Chapters 7-15, 8-38, 8-40 and 8-43 (2002).

The invention also relates to a method of the present invention wherein the buffers are pharmaceutically acceptable. “Pharmaceutically-acceptable” denotes an ingredient, such as a buffer ingredient, which upon administration to a patient does not provide a negative therapeutic event.

The invention also relates to a method of the present invention wherein the compound that may undergo a physical form change has a pKa. The pKa of a compound is determined experimentally or it can be calculated, such as with ACDLABS8.0 software.

The invention also relates to a method of the present invention wherein the compound that may undergo a physical form change is an active pharmaceutical ingredient (“API”). API's are commonly granulated using wet granulation methods.

The invention also relates to a method of the present invention wherein the pH is adjusted with a buffer having a pH either (a) about 2 units above the pKa for a basic API; or (b) about 2 units below the pKa of an acidic API.

The invention also relates to a method of the present invention wherein the buffer is of sufficient buffer capacity to maintain the pH after contacting material to be wet granulated. Merely adjusting the pH of an aqueous solution may not be sufficient if the ingredients of the wet granulation material have sufficient acid/base capacity to change the pH of the solution. If the API is a free acid, free base or a salt thereof, the pH may change significantly. The minimum buffer capacity that will be needed can be computed to be equal to the normality and mass of the API included in the wet granulation mixture.

The invention also relates to a method of the present invention wherein the buffer is added in a quantity to wet the formulation. In addition to the acid/base function of the buffer in the invention, the buffer solution needs also to function as a wetting agent. As such, the strength of the buffer must be balanced with the volume. Too weak a buffer may cause an excess volume to be needed to adjust the acid/base properties of the API but may form too dilute a granulation solution. Too strong a buffer may not provide enough solution to properly wet the granulation mixture. In general, fluid amounts of 3-5% are sufficient for wetting the solids, however a fluid amount of 5-40% is needed for granulation, depending on the excipients used.

The invention also relates to a method of the present invention wherein the API is a salt. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, example of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, ethanedisulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, β-hydroxybutyric, salicylic, galactaric and galacturonic acid. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the invention.

The invention also relates to a method of the present invention wherein the API has different physical forms. These forms may include polymorphs, hydrates or solvates.

The invention also relates to a method of the present invention wherein the API physical forms have different aqueous solubilities. For example, the compound can be a salt of an acid and its solubility may increase as the pH increases. If the pH of the water is controlled at pH where the solubility is minimum, then the rate of conversion will be slower and may not even take place due to the short time that the form is exposed to water. Alternatively, the compound can be a salt of a base and its solubility increases as the pH decreases.

The invention also relates to a method of maintaining anhydrous crystal form in an aqueous environment by adjusting the pH.

The invention also relates to a pharmaceutical composition comprising an API sensitive to water-related physical change further comprising a pH buffer which upon addition of water reduces the amount of water-related physical change. The composition may also include pH regulating agents. See Handbook of Pharmaceutical Excipients, 2^{nd} Ed., American Pharmaceutical Association, Wade and Waller eds. (1994).

The invention also relates to a method of preventing physical form change of an API during a wet granulation process comprising
A) determining if the API has different physical forms;
B) determining if the API physical forms have different aqueous solubilities;
C) determining the pKa of the API;
D) identifying a pharmaceutically-acceptable buffer having a pH either (a) about 2 units above the pKa for a basic API, or (b) about 2 units below the pKa for an acidic API; and
E) adding a pharmaceutically-acceptable buffer with capacity and volume sufficient to maintain the pH after contacting the API and to wet the excipients.

The wet granulation methods allow variation in the mixing process. For example the dry mix components can be blended followed by addition of the wetting agent buffer. The dry mix components, excluding the API, could first be wetted with the buffer, then the API could be added to the wetted mixture. Alternatively, the dry mix components can be blended into the wetting agent buffer. Further, the API could be suspended in the buffer and the suspension used to wet the remaining components of the granulation mixture. Further the wetted mixture is a granulation mixture.

The wet granulation process helps to form agglomerates of powders. These agglomerates are called “granules.” Wet granulated formulations may not need a binder. Instead, the drug itself can act as a binder so a formulation can be made with or without a binder.

The limit of detection of the present DSC and XRPD analytical instrumentation is about 1% to about 5%.

### Formulations

Also embraced within this invention is a class of pharmaceutical compositions comprising the granulated material in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. The daily dose can be administered in one to four doses per day.

For therapeutic purposes, the active compounds of this invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in a pharmaceutically acceptable polymer such as ethylcellulose, methylcellulose, hydroxypropylmethylcellulose (HPMC), hydroxyethylcellulose (HEC), acrylic acid ester, cellulose acetate phthalate, HEC phthalate, HPMC phthalate or other cellulosic polymers, or mixtures of polymers. The polymer coating can be applied using conventional methods, preferably a spray coating method employing bottom spray fluidized bed equipment.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, and flavoring agents.

Granulation is the process of adding water to a powder mixture with mixing until granules are formed. Wet granulation methods are known in the art and have been described in detail by Dilip M. Parikh (Handbook of Pharmaceutical Granulation Technology, 2^{nd} ed., edited by Marcel Dekker, 2005 ISBN:0824726472). The granulation step may be varied from 2 to 30 minutes, preferably 2 to 5 minutes. The lubrication step is the process of adding lubricant to the mixture; the lubrication step may be varied from 30 seconds to 20 minutes, preferably 3 to 8 minutes.

The disclosed process may be used to prepare solid dosage forms, particularly tablets, for medicinal administration.

Preferred diluents include: lactose, microcrystalline cellulose, calcium phosphate(s), mannitol, powdered cellulose, pregelatinized starch, and other suitable diluents. Especially preferred are lactose and microcrystalline cellulose, such as microcrystalline cellulose NF.

Wet granulated formulations may need to have an agent called a “binder,” which, in contact with water, swells or starts dissolving, forming a gel-like consistency. Traditionally, starch, starch paste, gelatin, and cellulosics such as hydroxypropylmethyl cellulose, hydroxyethyl cellulose, hydroxypropyl cellulose, polyvinyl pyrrolidone are used as binding agents in wet granulation formulations. (See, Remington's Pharmaceutical Sciences, 18^{th} ed., Mack Publishing Company: Easton, Pa., 1635-1636 (1990)). Microcrystalline cellulose, such as Avicel PH101, may be employed as a binder or compression aid in compositions prepared by dry granulation formulation, but microcrystalline cellulose functions primarily as a bulking agent in wet granulation formulations because the microcrystalline cellulose loses much of its binding properties upon wetting.

The disintegrant may be one of several modified starches or modified cellulose polymers, including croscarmellose sodium such as croscarmellose sodium NF Type A, commercially available under the trade name “Ac-di-sol”.

Lubricants include magnesium stearate, calcium stearate, stearic acid, surface active agents such as sodium lauryl sulfate, propylene glycol, sodium dodecane sulfonate, sodium oleate sulfonate, and sodium laurate mixed with stearates and talc, sodium stearyl fumerate, and other known lubricants.

The invention will now be further described with reference to the following non-limiting examples.

### EXAMPLE 1

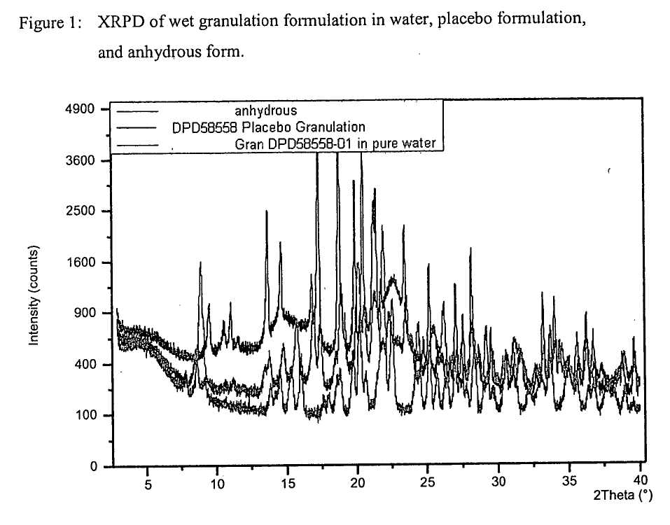

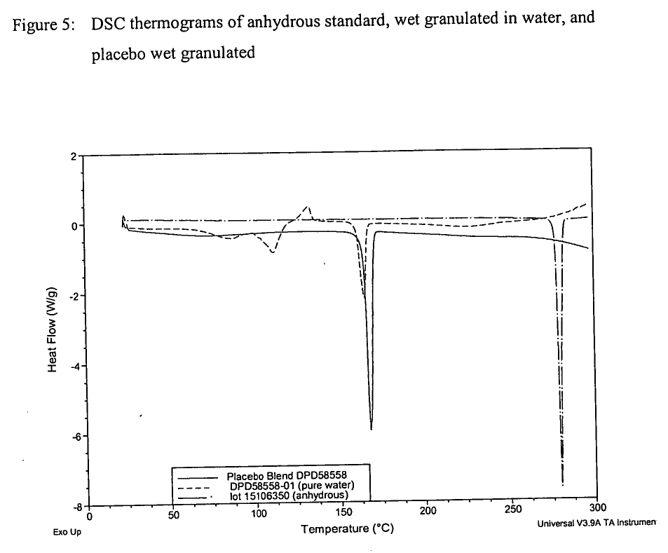

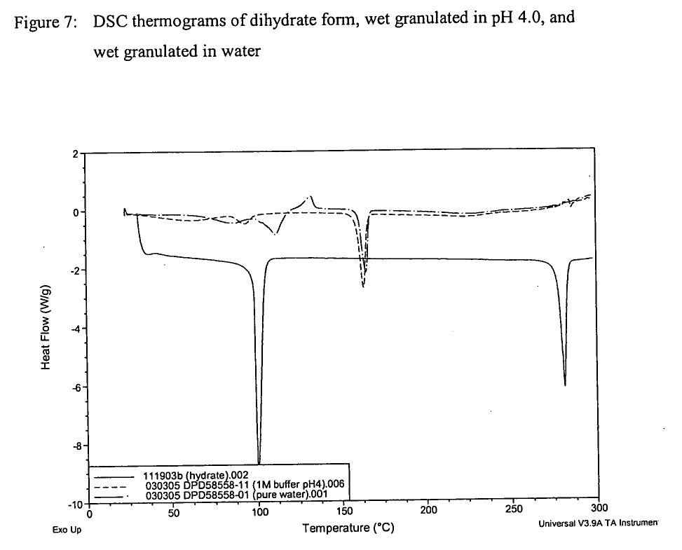

A wet granulation excipients mixture of mannitol (Manogem EZ, 67.05 g, 26.82%) microcrystalline cellulose NF (Avicel PHl102, 50 g, 20%), povidone (K29/32 USP, 7.5 g, 3%), sodium starch glycolate (Explotab, 5 g, 2%) and model Compound A (109.2 g, 43.68%) was passed through a 40 mesh screen and added to a high shear granulator (Dionsa 2L bowl). The pKa of Compound A is around 6.6, the compound exists in water as the free acid (99.75%) at pH 4.0 and the solubility in water at pH 4 is very low (less than 15 μg/mL). The granulator was maintained at a constant impeller speed (550 RPM) and chopper speed (1500 RPM) and mixed for 1 min. De-ionized water with no pH adjustment (measured pH-6.17) (46.2 g, 19.35%) was sprayed on the mixture at the rate of 30 g/min (or 16.4 g/min) using a Masterflex pump for about 3 minutes to wet the ingredients. The granules were loaded onto a drying tray for tray bed drying. The mixture was dried in a forced air confection oven at 60° C. for about 2 hours or until the target loss on drying (LOD) of 2% was achieved. The material was analyzed for hydrate formation. The XRPD results are shown in FIG. 1. The predominant form appears to be the dihydrate. The DSC results are shown in FIGS. 5 and 7.

### EXAMPLE 2

The blend was prepared essentially as described in Example 1 except de-ionized water adjusted to pH 2 was used to wet the ingredients.

### EXAMPLE 3

The blend was prepared essentially as described in Example 1 except de-ionized water adjusted to pH 5 was used to wet the ingredients.

### EXAMPLE 4

The blend was prepared essentially as described in Example 1 except de-ionized water adjusted to pH 9 was used to wet the ingredients.

### EXAMPLE 5

The blend was prepared essentially as described in Example 1 except 0.2 M sodium acetate buffer at pH 4 was used to wet the ingredients.

### EXAMPLE 6

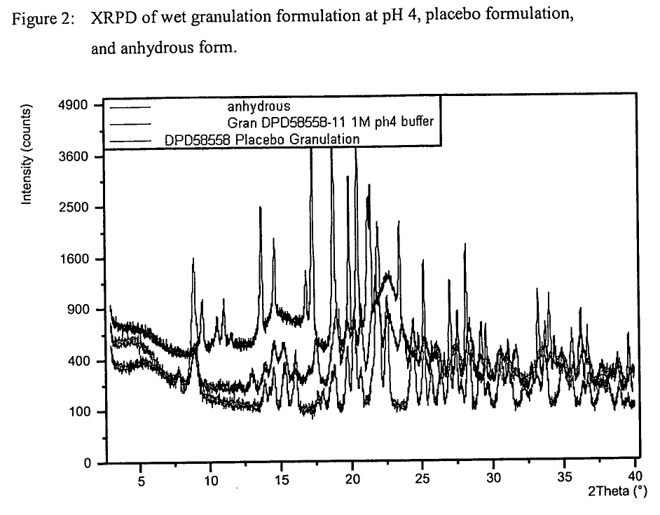

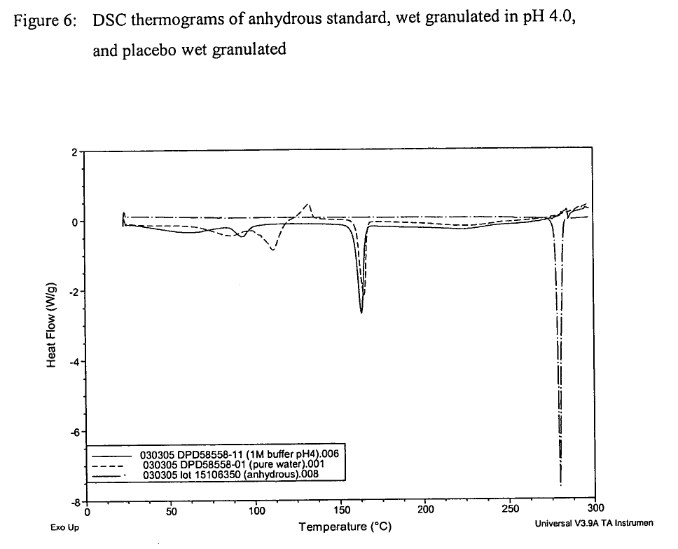

The blend was prepared essentially as described in Example 1 except 1.0 M sodium acetate buffer at pH 4 was used to wet the ingredients. The XRPD results are shown in FIG. 2. The predominant form appears to be the anhydrous form. The DSC results are shown in FIGS. 6 and 7.

Compound A, 3-chloro-2-methyl-N-(4-(2-(3-oxo-4-morpholinyl)ethyl)-1,3-thiazol-2-yl)benzenesulfonamide, was prepared essentially as described in U.S. Publication No. 2004/0224996A1 and converted to the potassium salt.

The foregoing is merely illustrative of the invention and is not intended to limit the invention to the disclosed methods or processes. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

All mentioned references, patents, applications and publications, are hereby incorporated by reference in their entirety, as if here written.

What is claimed is:

1. A method of preventing a physical form change in a compound during a wet granulation process comprising a pH adjustment.

2. The method of claim 1 wherein the pH adjustment is made with an acidic or basic buffer.

3. The method of claim 2 wherein the acidic buffer is selected from glutamate, citrate, and acetate.

4. The method of claim 1 wherein the compound is an API.

5. The method of claim 4 wherein the API is a salt.

6. The method of claim 4 wherein the API has different physical forms.

7. The method of claim 4 wherein the API physical forms have different aqueous solubilities.

8. The method of claim 7 wherein the physical form is a hydrate.

9. The method of claim 4 wherein the pH is adjusted with a buffer having a pH either (a) about 2 units above the pKa for a basic compound, or (b) about 2 units below the pKa for an acidic compound.

10. The method of claim 9 wherein the buffer is of sufficient buffer capacity to maintain the pH after contacting the API.

11. The method of claim 9 wherein the buffer is added in a quantity to wet the formulation.

12. The method of claim 2 wherein the buffer is pharmaceutically acceptable.

13. A method of maintaining anhydrous crystal form of a compound in an aqueous environment by adjusting the pH.

14. A pharmaceutical composition comprising an API sensitive to water-related physical change further comprising a pH buffer which upon addition of water reduces the amount of water-related physical change.

15. The method of claim 1 wherein the compound is in a granulation fluid.

16. A method of preventing physical form change of an API during a wet granulation process comprising
A) determining if the API has different physical forms;
B) determining if the API physical forms have different aqueous solubilities;
C) determining the pKa of the API;
D) identifying a pharmaceutically-acceptable buffer having a pH either (a) about 2 units above the pKa for a basic API, or (b) about 2 units below the pKa for an acidic API; and
E) adding a pharmaceutically-acceptable buffer with capacity and volume sufficient to maintain the pH after contacting the API and to wet the excipients.
